(19) United States
(12) Patent Application Publication
(10) Pub. No.: US 2015/0265450 A1
(43) Pub. Date: Sep. 24, 2015

(54) SOFT SPLINT

(71) Applicant: Maverick Sports Medicine, Inc., Redmond, WA (US)

(72) Inventor: Jeffrey R. Rodgers, Bellevue, WA (US)

(21) Appl. No.: 14/218,226

(22) Filed: Mar. 18, 2014

(51) Int. Cl. A61F 5/01; A61H 1/00

(52) U.S. Cl. CPC A61F 5/0127; A61H 1/008

(57) ABSTRACT

A soft splint having an insole formed of rigid material and having a top, a bottom, and a toe wedge section, a sock having an open top, an open forefoot and toe, a bottom, and an interior space, the sock having a bottom attached to the bottom of the insole with a portion of the top of the insole located in the interior space of the sock, and first and second flexible straps, each strap having a first end attached to the bottom of the insole and a second end configured to attach to the open top of the sock.

### BACKGROUND

1. Technical Field

The present disclosure pertains to orthopedic devices for the lower leg and, more particularly, to a soft splint that combines a compression sleeve with a stability platform for use in alleviating pain and tightness in the arch and Achilles tendon resulting from plantar fasciitis or Achilles tendonitis or both.

2. Description of the Related Art

Discomfort and pain in the foot and lower leg is often attributed to injury of the plantar fascia. The plantar fascia is a thick band of fibrous connective tissue that runs from the heel bone (calcaneus) to the ball of the foot. It serves to maintain the arch of the foot by pulling the heel towards the ball. In addition, the Achilles tendon attaches to the calcaneus and serves to connect the muscles in the calf to the foot.

Active inflammation of the plantar fascia (plantar fasciitis) can also impact the Achilles tendon, and likewise force exerted through the Achilles tendon can impact the plantar fascia. This results in further damage to the surrounding tissue, and repetitive injury can result in bone spurs. Those who experience plantar fasciitis find it painful and difficult to stand, walk, and engage in recreational activities.

Treatment of plantar fasciitis and inflammation of the Achilles tendon can include one or a combination of modalities, such as rest and inactivity, taking of anti-inflammatory medications, stretching, exercise, massage, acupuncture, the use of orthotics and splints, and, as a last resort, surgery. A variety of orthotics are available with and without prescription, including wraps, compression socks, shoe inserts, heel cups, static progressive stretch braces, and hard and soft splints. In addition, night splints can be employed for several hours or more to maintain a stretched condition of the calf and plantar fascia during sleep.

### BRIEF SUMMARY

The present disclosure provides a device that is designed to alleviate pain and tightness in the arch and Achilles tendon. It combines the benefits of compression, such as from a compression sleeve, and the stability of a traditional night splint.

In accordance with one aspect of the present disclosure, a soft splint is provided that includes an insole formed of rigid material and having a top, a bottom, and a toe wedge section; a sock having an open top, an open forefoot and toe, a bottom, and an interior space, the sock having a bottom attached to the bottom of the insole with a portion of the top of the insole located in the interior space of the sock; and first and second flexible straps, each strap having a first end attached to the bottom of the insole and a second end configured to attach to the open top of the sock.

In accordance with another aspect of the present disclosure, the insole of the splint has a forefoot area, and the first end of each strap is attached to the forefoot area of the insole. Ideally, each of the first and second straps includes a section of elastic material having first and second sides and first and second sections of inelastic material, the section of elastic material positioned between the first and second sections of inelastic material.

In accordance with a further aspect of the present disclosure, the toe wedge section includes a toe wedge sleeve and a toe wedge piece made from foam material placed within the toe wedge sleeve and positioned on top of the insole and configured to support the user's forefoot and toes.

In accordance with still yet another aspect of the present disclosure, a pair of rings or loops are provided at the open top of the sock and configured to enable a user to feed the first and second flexible straps there-through and pull the insole's forefoot area toward the user's knee in a dorsiflexion angle and point the user's toes towards their knee.

In accordance with another aspect of the present disclosure, a soft splint is provided that has an insole with a forefoot area; a sock having a top and a stretchable strap with hook-and-loop closure, the strap configured to wrap around a user's leg just below knee and at the top of the sock; and a pair of rings or loops at the top of the sock and made from a rigid material, each ring or loop configured to attach to a respective side of the strap after the strap is wrapped a user's leg and to allow additional straps to feed there-through.

In accordance with yet a further aspect of the present disclosure, each of the first and second flexible side straps are sized and shaped to enable varying an attachment point of the strap to the body.

### BRIEF DESCRIPTION OF THE SEVERAL VIEWS OF THE DRAWINGS

The foregoing features and advantages of the present disclosure will be more readily appreciated as the same become better understood from the accompanying drawings, wherein:

FIG. 1 is an isometric view of a soft splint formed in accordance with the present disclosure;

FIG. 2 is a partially exploded view of the soft splint of FIG. 1;

FIG. 3 is a top plan view of the soft splint of FIG. 1;

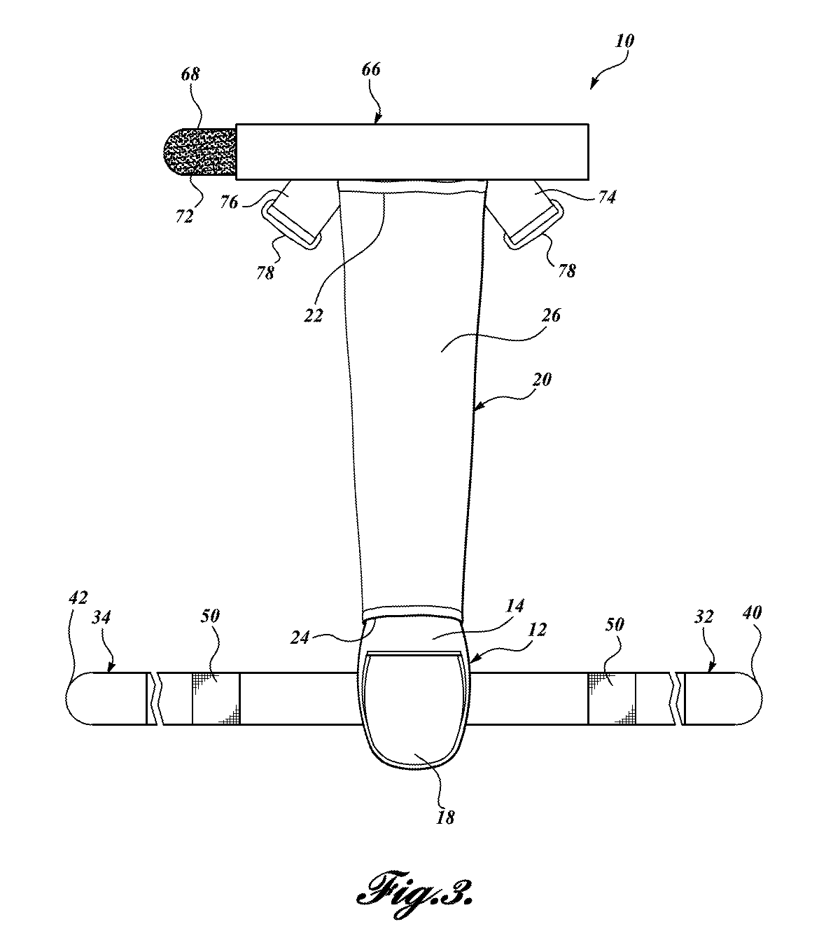

FIG. 4 is a bottom plan view of the soft splint of FIG. 1; and

FIG. 5 is an isometric view of the soft splint of FIG. 1 as worn on the foot and leg.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or components or both associated with the splints, including but not limited to insoles, cushions, straps, and connecting members such as buckles and hook-and-loop fasters have not been shown or described in order to avoid unnecessarily obscuring descriptions of the embodiments.

Unless the context requires otherwise, throughout the specification and claims that follow, the word “comprise” and variations thereof, such as “comprises” and “comprising” are to be construed in an open inclusive sense, that is, as “including, but not limited to.” The foregoing applies equally to the words “including” and “having.”

Reference throughout this description to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same embodiment. Furthermore, the particular features, structures, or characteristics may be combined in any suitable manner in one or more embodiments.

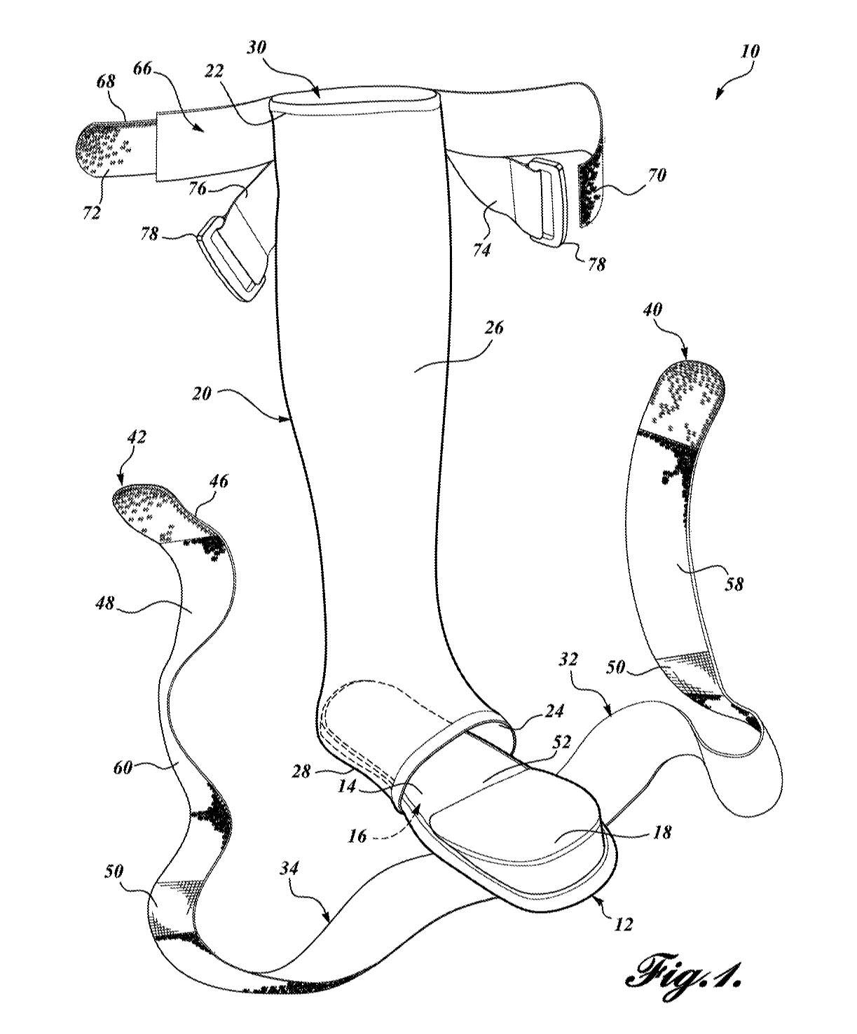

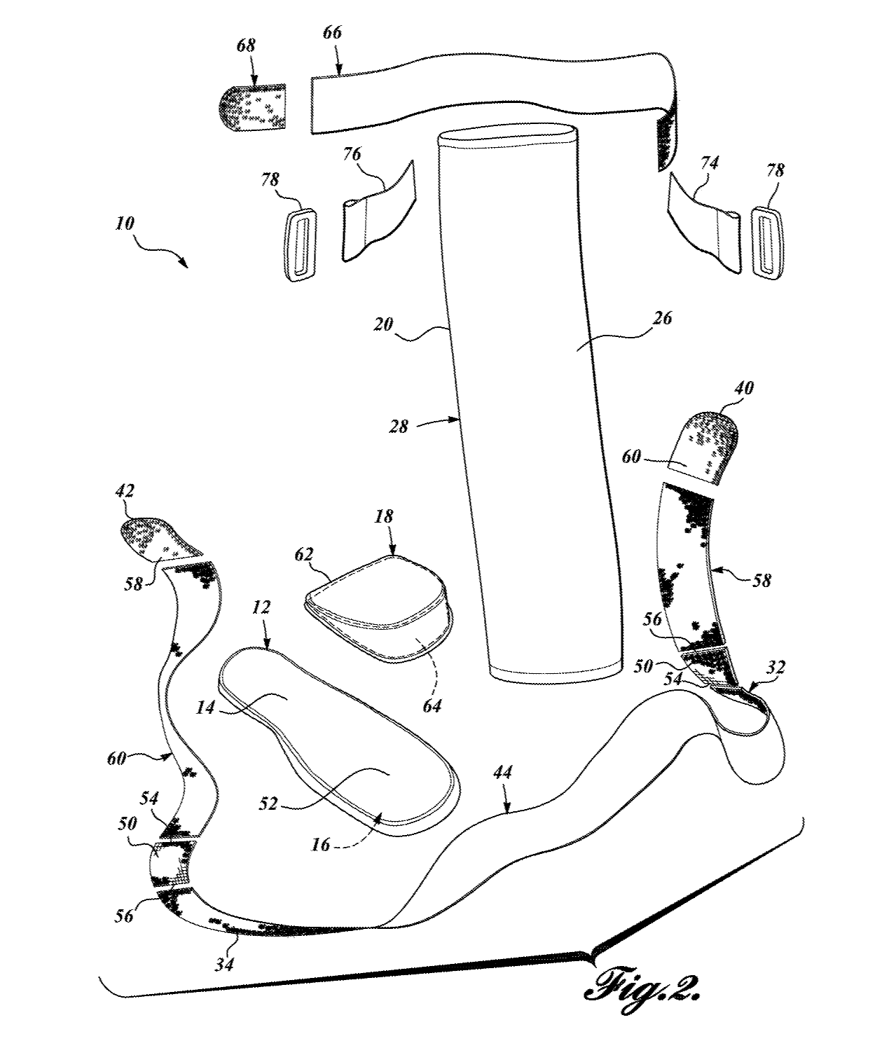

Referring initially to FIGS. 1 and 2, shown therein is a soft splint 10 having an insole 12 formed of rigid material and having a top 14, a bottom 16, and a toe wedge section 18. Attached to the insole 12 is a sock 20 having an open top 22, an open forefoot and toe 24, a sidewall 26 with a bottom section 28, and an interior space 30. The bottom section 28 of the sock is attached to the bottom 16 of the insole 12 with a portion of the top 14 of the insole 12 located in the interior space 30 of the sock 20. Preferably, the insole 12 extends about half of its length into the sock 20.

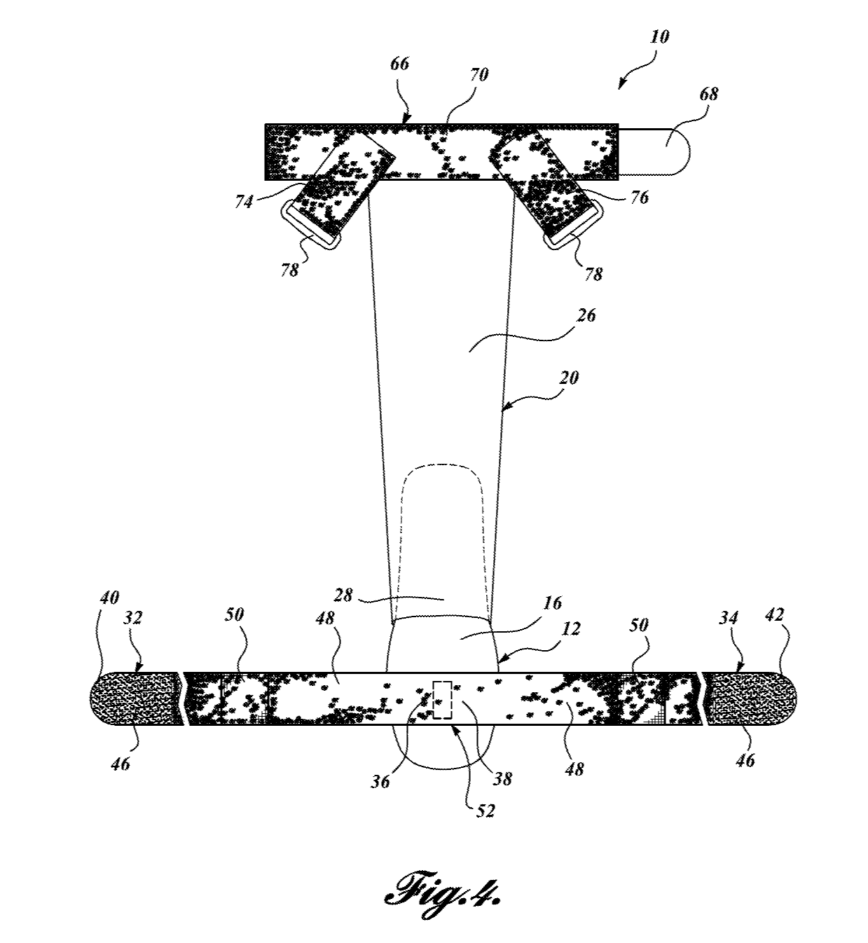

First and second flexible straps 32, 34 are provided, each strap 32, 34 having a respective first end 36, 38 attached to the bottom 16 of the insole 12 as shown in FIG. 4, and a second end 40, 42 configured to attach to the open top 22 of the sock 20 as described in more detail herein. Preferably, hook-and-loop fasteners are used to allow the second ends 40, 42 to fold back and attach to their respective straps 32, 34. For example, the first and second ends 40, 42 have a respective terminal end section covered with the hook portion 46 and the reverse side has a section covered with the loop portion 48. Preferably, the remainder of the straps 32, 34 are covered with the loop portion to provide maximum adjustability as shown in the bottom view of FIG. 4.

It is to be understood that the first and second straps 36, 38 can be formed from a single piece of flexible filament 44 as shown in FIG. 2, which preferably is not stretchable or elastic yet is still flexible in order to bend. In addition, each of the first and second straps 36, 38 includes a stretchable segment 50. This facilitates exerting a continuous amount of tension on the straps 36, 38, regardless of the position of the user's foot.

The insole 12 of the splint 10 has a forefoot area 52, and the first end 36, 38 of each strap 32, 34 is attached to the forefoot area 52 of the insole 12 so the straps 32, 34 extend laterally outward and away from their respective side of the insole 12. As described above, each of the first and second straps 32, 34 includes the segment of elastic material 50 having first and second side edges 54, 56. In addition, the straps 32, 34 include first and second sections 58, 60 of inelastic material, the segment of elastic material 52 is positioned between the first and second sections 58, 60 of inelastic material as shown in FIG. 2. Each of the elastic segments 50 have their first and second side edges 54, 56 attached to their respective inelastic sections 58, 60 preferably by stitching. Similarly, the straps 32, 34 are attached to the bottom 16 of the insole 12 at the forefoot area 52 with stitching.

The toe wedge section 18 includes a toe wedge sleeve 62 and a toe wedge piece 64 made from foam material placed within the toe wedge sleeve 62 and positioned on the top 14 of the insole 12. The toe wedge 18 section is configured to support the user's forefoot and toes. Preferably the sleeve 62 is stitched to the insole 12 in a conventional manner.

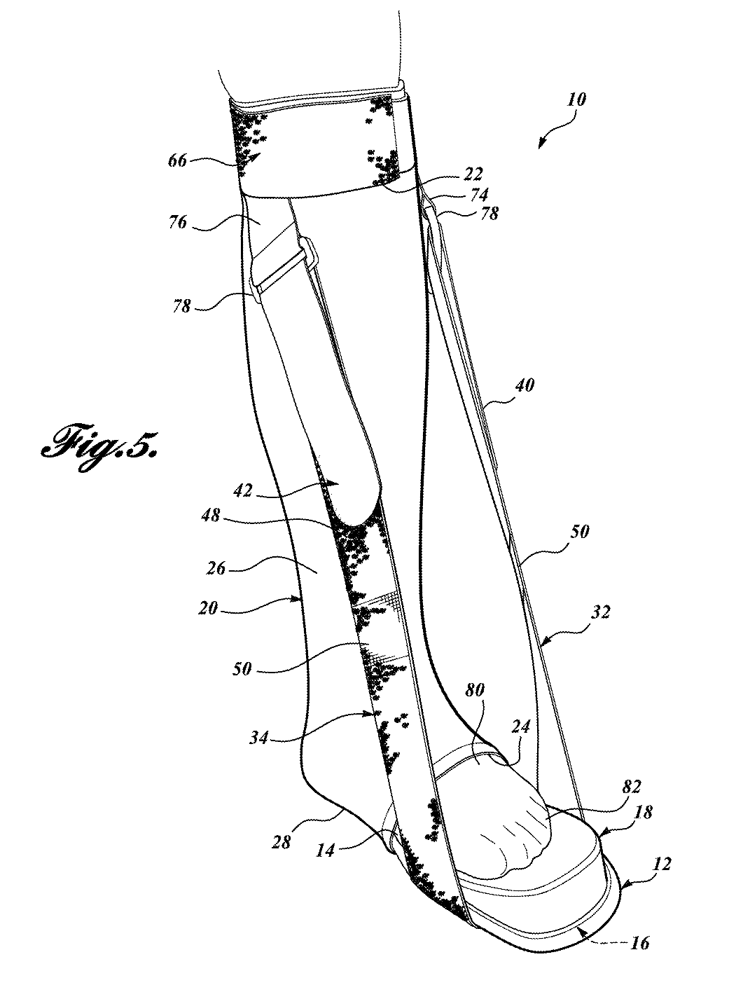

Attached across a portion of the top 22 of the sock 20 is a flexible and stretchable top strap 66 having a first side covered with one portion of a hook-and-loop fastener, and a tab 68 extending from one side of the top strap 66 with the other portion of the hook-and-loop fastener on one side of the tab 68. Preferably the first side of the top strap 66 is covered with the loop portion 70 and the one side of the tab 68 has the hook portion 72. The top strap 66 is configured to wrap around a user's leg just below knee and at the top of the sock 20 and attach to itself with the hook-and-loop fasterners to hold the sock in place on the user's leg as shown in FIG. 5.

Extending at an angle from the top strap 66 on each side at the top 22 of the sock 20 are first and second ring support segments 74, 76 with a first end attached to the top strap 66 and a second end having a ring or loop 78 attached thereto. These rings or loops 78 are provided adjacent the open top 22 of the sock 20 and are configured to enable a user to feed the first and second flexible straps 32, 34 there-through and pull the insole's forefoot area 52 toward the user's knee in a dorsiflexion angle and point the user's toes towards their knee.

FIG. 5 shows the soft splint 10 in use on a user's foot 80. The user has placed their foot 80 through the open top 22 of the sock 20 and extended it out through the open forefoot and toe 24 so that the bottom of the foot 80 rests on the insole 12 and the toes 82 and a portion of the ball of the foot 80 are supported on the toe wedge 18. The top strap 66 is stretched around the user's leg just below the knee and held in place with the hooks 72 on the tab 68 engaging the loops 70 on the one side of the top strap 66. Each of the first and second straps 32, 34 are fed through their respective ring or loop 78 and folded back onto itself so that the loops 46 on the respective ends 40, 42 engage the hooks 48 on the straps 32, 24. The inelastic segments 58, 60 on each strap 32, 34 may be pulled tight and the elastic segments 50 on each strap 32, 34 can be stretched in order to provide tension on the insole 12 and urge the toes 82 of the user's foot 80 to move toward the user's knee. This tension stretches the Achilles tendon and also stretches the plantar fascia.

The various aspects of the present disclosure described above can be combined to provide further embodiments. In addition, various features of the present disclosure can be modified, if necessary or desired, to provide yet further embodiments.

These and other changes can be made in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A soft splint, comprising:
an insole formed of rigid material and having a top, a bottom, and a toe wedge section;
a sock having an open top, an open forefoot and toe, a bottom, and an interior space, the sock having a bottom attached to the bottom of the insole with a portion of the top of the insole located in the interior space of the sock; and
first and second flexible straps, each strap having a first end attached to the bottom of the insole and a second end configured to attach to the open top of the sock.

2. The splint of claim 1 wherein the insole has a forefoot area, and the first end of each strap is attached to the forefoot area of the insole.

3. The splint of claim 2 wherein each of the first and second straps comprise a section of elastic material having first and second sides and first and second sections of inelastic material, the section of elastic material positioned between the first and second sections of inelastic material.

4. The splint of claim 1 wherein the toe wedge section comprises a toe wedge sleeve and a toe wedge piece made from foam material placed within the toe wedge sleeve and positioned on top of the insole and configured to support the user's forefoot and toes.

5. The splint of claim 1 where the sock is made from a multidirectional, breathable material.

6. The splint of claim 1 wherein each of the first and second flexible side straps are configured to enable varying an attachment point of the strap to the body.

7. The splint of claim 2, comprising a pair of rings or loops at the open top of the sock and configured to enable a user to feed the first and second flexible straps there-through and pull the insole's forefoot area toward the user's knee in a dorsiflexion angle and point the user's toes towards their knee.

8. A soft splint, comprising:
an insole having a forefoot area;
a sock having a top and a stretchable strap with hook-and-loop closure, the strap configured to wrap around a user's leg just below knee and at the top of the sock; and
a pair of rings or loops at the top of the sock and made from a rigid material, each ring or loop configured to attach to a respective side of the strap after the strap is wrapped around the user's leg and to allow additional straps to feed there-through.

9. The splint of claim 6 wherein each of the first and second flexible side straps are configured to enable varying an attachment point of the strap to the body.

10. The splint of claim 6 wherein the pair of rings or loops are configured to enable a user to feed the first and second flexible straps there-through and pull the insole's forefoot area toward the user's knee in a dorsiflexion angle and point the user's toes towards their knee.
